(19) United States
(12) Patent Application Publication
(10) Pub. No.: US 2008/0096274 A1
(43) Pub. Date: Apr. 24, 2008

(54) Recombinant alphavirus vectors and methods of using same

(76) Inventor: Guangpu Li, Edmond, OK (US)

Correspondence Address: DUNLAP CODDING & ROGERS, P.C., PO BOX 16370, OKLAHOMA CITY, OK, 73113, US

(21) Appl. No.: 11/977,085

(22) Filed: Oct. 23, 2007

Related U.S. Application Data
(60) Provisional application No. 60/853,983, filed on Oct. 24, 2006.

(51) Int. Cl. C07H 21/04; C12N 15/00; C12N 5/02

(52) U.S. Cl. 435/377; 435/320.1; 536/23.4

(57) ABSTRACT

The present invention describes nucleic acid segments, recombinant alphavirus vectors, and recombinant alphavirus particles that include a Sindbis viral vector. The Sindbis viral vector includes a nucleic acid segment encoding a fusion protein comprising a Sindbis virus nonstructural protein and a protein or peptide of interest, wherein the production of the fusion protein does not affect viral replication or infection. The protein or peptide of interest may be a marker, diagnostic, or therapeutic protein or peptide. Methods of using such recombinant alphavirus particles to kill tumor cells is also described herein.

### CROSS REFERENCE TO RELATED APPLICATIONS

This application claims benefit under 35 U.S.C. 119(e) of U.S. Ser. No. 60/853,983, filed Oct. 24, 2006; the entire contents of which has been expressly incorporated herein by reference in its entirety.

### STATEMENT REGARDING FEDERALLY SPONSORED RESEARCH OR DEVELOPMENT

Not Applicable.

### BACKGROUND OF THE INVENTION

Alphaviruses comprise a set of genetically, structurally, and serologically related arthropod-borne viruses of the Togaviridae family. These viruses are distributed worldwide, and persist in nature through a mosquito to vertebrate cycle. Birds, rodents, horses, primates, and humans are among the defined alphavirus vertebrate reservoir/hosts.

Sindbis virus is the prototype member of the Alphavirus genus of the Togaviridae family. Its replication strategy after infection of cells has been well characterized in chicken embryo fibroblasts (CEF) and baby hamster kidney (BHK) cells, where Sindbis virus grows rapidly and to high titer, and serves as a model for other alphaviruses. Briefly, the genome from Sindbis virus (like other alphaviruses) is an approximately 12 kb single-stranded positive-sense RNA molecule which is capped and polyadenylated, and contained within a virus-encoded capsid protein shell. The nucleocapsid is further surrounded by a host-derived lipid envelope into which two viral-specific glycoproteins, E1 and E2, are inserted and anchored to the nucleocapsid. Certain alphaviruses (e.g., SF) also maintain an additional protein, E3, which is a cleavage product of the E2 precursor protein, PE2. After virus particle absorption to target cells, penetration, and uncoating of the nucleocapsid to release viral genomic RNA into the cytoplasm, the replicative process is initiated by translation of the nonstructural proteins (nsPs) from the 5′ two-thirds of the viral genome. The four nsPs (nsP1-nsP4) are translated directly from the genomic RNA template as one of two polyproteins (nsP123 or nsP1234), and processed post-translationally into monomeric units by an active protease in the C-terminal domain nsP2. A leaky opal (UGA) codon present between nsP3 and nsP4 of most alphaviruses accounts for a 10 to 20% abundance of the nsP1234 polyprotein, as compared to the nsP123 polyprotein. Both of the nonstructural polyproteins and their derived monomeric units may participate in the RNA replicative process, which involves binding to the conserved nucleotide sequence elements (CSEs) present at the 5′ and 3′ ends, and a junction region subgenomic promoter located internally in the genome.

The positive strand genomic RNA serves as template for the nsP-catalyzed synthesis of a full-length complementary negative strand. Synthesis of the complementary negative strand is catalyzed after binding of the nsP complex to the 3′ terminal CSE of the positive strand genomic RNA. The negative strand, in turn, serves as template for the synthesis of additional positive strand genomic RNA and an abundantly expressed 26S subgenomic RNA, initiated internally at the junction region promoter. Synthesis of additional positive strand genomic RNA occurs after binding of the nsP complex to the 3′ terminal CSE of the complementary negative strand genomic RNA template. Synthesis of the subgenomic mRNA from the negative strand genomic RNA template, is initiated from the junction region promoter. Thus, the 5′ end and junction region CSEs of the positive strand genomic RNA are functional only after they are transcribed into the negative strand genomic RNA complement (i.e., the 5′ end CSE is functional when it is the 3′ end of the genomic negative stranded complement). The structural proteins (sPs) are translated from the subgenomic 26S RNA, which represents the 3′ one-third of the genome, and like the nsPs, are processed post-translationally into the individual proteins.

Several groups have suggested utilizing certain members of the alphavirus genus as an expression vector, including, for example, Sindbis virus (Xiong et al., Science 243:1188-1191, 1989; Hahn et al., Proc. Natl. Acad. Sci. USA 89:2679-2683, 1992; Dubensky et al., J. Virol. 70:508-519, 1996), Semliki Forest virus (Liljestrom, Bio/Technology 9:1356-1361, 1991), and Venezuelan Equine Encephalitis virus (Davis et al., J. Cell. Biochem. Suppl. 19A:10, 1995). In addition, one group has suggested using alphavirus-derived vectors for the delivery of therapeutic genes in vivo. One difficulty, however, with the above-referenced vectors is that inhibition of host cell-directed macromolecular synthesis (i.e., protein or RNA synthesis) begins within a few hours after infection and cytopathic effects (CPE) occur within 12 to 16 hours post infection (hpi). Inhibition and shutoff of host cell protein synthesis begins within 2 hpi in BHK cells infected with recombinant viral particles, in the presence or absence of structural protein expression, suggesting that the early events after virus infection (e.g., synthesis of nsPs and minus strand RNA) may directly influence the inhibition of host cell protein synthesis and subsequent development of CPE and cell death.

Further descriptions of recombinant Sindbis viral vectors can be found in U.S. Pat. Nos. 7,005,275, issued to Renner et al., on Feb. 28, 2006; 6,635,476 issued to Murphy on Oct. 21, 2003; 6,465,634, issued to Dubensky et al., on Oct. 15, 2002; 6,376,236, issued to Dubensky et al., on Apr. 23, 2002; 6,329,201, issued to Polo et al., on Dec. 11, 2001; 5,843,723, issued to Dubensky et al., on Dec. 1, 1998; 5,789,245, issued to Dubensky et al., on Aug. 4, 1998; and 5,091,309, issued to Schlesinger et al., on Feb. 25, 1992; the contents of each of which are hereby expressly incorporated herein by reference.

The present invention provides recombinant vectors for use in a variety of applications, including for example, therapeutic and diagnostic uses, including use as oncolytic agents, as well as methods of producing and using same.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 illustrates single-step growth curves of Toto1101/GFP and Toto1101 viruses. Virus samples were collected at the indicated times and titered by plaque assays. The bar graph shows the virus accumulation over a 24-hour period. The data represent the mean of duplicate samples and were reproducible in two independent experiments.

FIG. 2 illustrates post-translational processing of nsP3-GFP. BHK cells infected with either the Toto1101/GFP virus (indicated) or the parental Toto1101 virus (indicated) were pulse-labeled with [³⁵S]methionine for 15 min, followed by different times of chase as indicated in normal growth medium. Cells were lysed in 1% SDS and the lysates were immunoprecipitated with a nsP3-specific antibody as previously described, followed by SDS-PAGE and autoradiography. Note the mobility shift of both nsP3 and nsP3-GFP after the chase, which is indicative of post-translational phosphorylation. The nsP3 and its precursors are indicated on the left, while the nsP3-GFP and its precursors are indicated on the right. A possible degradation product of nsP3-GFP, referred to as nsP3x, is also indicated on the right. Molecular weight standards (in kDa) are indicated in the middle.

FIG. 3 illustrates localization of nsP3-GFP during early infection. BHK cells grown on cover slips were infected with the Toto1101/GFP virus and incubated at 37° C. At 2 h post-infection, the cells were fixed with 4% paraformaldehyde (Electron Microscopy Sciences), mounted on glass slides (Fishser Scientific) and viewed via a Leica confocal fluorescence microscope. Shown are two confocal images of different fields. Panel A shows free single cells, while panel B shows two or more cell clusters. Arrows indicate plasma membrane localized nsP3-GFP.

FIG. 4 illustrates localization of nsP3-GFP during late infection. The experimental procedure was identical to the FIG. 3 legend, except that the cells were fixed and processed for microscopy at later times. Panels A and B show confocal images taken at 3 and 10 h post-infection. Bar=20 μm.

FIG. 5 illustrates that EGF stimulates Sindbis virus replication in PC12 cells. PC12 cells were either treated or not treated with EGF for 12 hours, followed by infection with the Toto1101/GFP virus. Virus samples were collected at the indicated times and titered by plaque assays. The bar graph shows the virus accumulation over a 24-hour period. The data represent the mean of duplicate samples and were reproducible in two independent experiments.

FIG. 6 illustrates that Sindbis virus preferentially replicates in Ras-transformed NIH 3T3 cells. A NIH 3T3 cell line stably transfected with the oncogenic v-H-Ras gene and a control NIH 3T3 cell line transfected with the empty vector were infected with the Toto1101/GFP virus. Virus samples were collected at the indicated times and titered by plaque assays. The bar graph shows the virus accumulation over a 48-hour period. The data represent the mean of duplicate samples and were reproducible in two independent experiments.

### DETAILED DESCRIPTION OF THE PRESENT INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “polypeptide” as used herein is a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “recombinant” in the context of polypeptide coding regions and the polypeptides encoded by such coding regions refers to non-native products wherein the coding regions, and typically the expression thereof, have been manipulated in vitro by man to differ from their occurrence in nature. The polypeptides utilized in the methods of the present invention may be produced in a number of different recombinant systems known in the art, including but not limited to, archeal, prokaryotic, or eukaryotic systems. For expression in an appropriate expression system, the desired viral capsid polypeptide coding regions are operably linked into an expression vector and introduced into a host cell to enable expression. The coding region with the appropriate regulatory regions will be provided in proper orientation and reading frame to allow for expression. Methods for gene construction are known in the art. See, in particular, Molecular Cloning, A Laboratory Manual, Sambrook et al, eds., Cold Spring Harbor Laboratory, Second Edition, Cold Spring Harbor, N.Y. (1989) and the references cited therein.

As used herein, the term “alphavirus” refers to any of the RNA viruses included within the genus Alphavirus. Descriptions of the members of this genus are contained in Strauss and Strauss, Microbiol. Rev., 58:491-562 (1994). Examples of alphaviruses include Aura virus, Bebaru virus, Cabassou virus, Chikungunya virus, Eastern equine encephalomyelitis virus, Fort morgan virus, Getah virus, Kyzylagach virus, Mayoaro virus, Middleburg virus, Mucambo virus, Ndumu virus, Pixuna virus, Tonate virus, Triniti virus, Una virus, Western equine encephalomyelitis virus, Whataroa virus, Sindbis virus (SIN), Semliki forest virus (SFV), Venezuelan equine encephalomyelitis virus (VEE), and Ross River virus.

As used herein, when the term “purified” is used in reference to a molecule, it means that the concentration of the molecule being purified has been increased relative to molecules associated with it in its natural environment. Naturally associated molecules include proteins, nucleic acids, lipids and sugars but generally do not include water, buffers, and reagents added to maintain the integrity or facilitate the purification of the molecule being purified. For example, even if mRNA is diluted with an aqueous solvent during oligo dT column chromatography, mRNA molecules are purified by this chromatography if naturally associated nucleic acids and other biological molecules do not bind to the column and are separated from the subject mRNA molecules.

As used herein, when the term “isolated” is used in reference to a molecule, the term means that the molecule has been removed from its native environment. For example, a polynucleotide or a polypeptide naturally present in a living animal is not “isolated,” but the same polynucleotide or polypeptide separated from the coexisting materials of its natural state is “isolated.” Further, recombinant DNA molecules contained in a vector are considered isolated for the purposes of the present invention. Isolated RNA molecules include in vivo or in vitro RNA replication products of DNA and RNA molecules. Isolated nucleic acid molecules further include synthetically produced molecules. Additionally, vector molecules contained in recombinant host cells are also isolated. Thus, not all “isolated” molecules need be “purified.”

As used herein, the phrase “individual” refers to multicellular organisms and includes both plants and animals. Preferred multicellular organisms are animals, more preferred are vertebrates, even more preferred are mammals, and most preferred are humans.

As used herein, the phrase “cis-acting” sequence refers to nucleic acid sequences to which a replicase binds to catalyze the RNA-dependent replication of RNA molecules. These replication events result in the replication of the full-length and partial RNA molecules and, thus, the alpahvirus subgenomic promoter is also a “cis-acting” sequence. Cis-acting sequences may be located at or near the 5′ end, 3′ end, or both ends of a nucleic acid molecule, as well as internally.

As used herein, the phrase “RNA-Dependent RNA polymerase” refers to a polymerase which catalyzes the production of an RNA molecule from another RNA molecule. This term is used herein synonymously with the term “replicase.”

As used herein, the term “transcription” refers to the production of RNA molecules from DNA templates catalyzed by RNA polymerases.

As used herein, the phrase “RNA-dependent RNA replication event” refers to processes which result in the formation of an RNA molecule using an RNA molecule as a template.

As used herein, the term “vector” refers to an agent (e.g., a plasmid or virus) used to transmit genetic material to a host cell. A vector may be composed of either DNA or RNA.

As used herein, the term “heterologous sequence” refers to a second nucleotide sequence present in a vector of the invention. The term “heterologous sequence” also refers to any amino acid or RNA sequence encoded by a heterologous DNA sequence contained in a vector of the invention. Heterologous nucleotide sequences can encode proteins or RNA molecules normally expressed in the cell type in which they are present or molecules not normally expressed therein (e.g., Sindbis structural or nonstructural proteins).

As used herein, the phrase “untranslated RNA” refers to an RNA sequence or molecule which does not encode an open reading frame or encodes an open reading frame, or portion thereof, but in a format in which an amino acid sequence will not be produced (e.g., no initiation codon is present). Examples of such molecules are tRNA molecules, rRNA molecules, and ribozymes. Antisense RNA may be untranslated but, in some instances (see Example 11), antisense sequences can be converted to a translatable sense strand from which a polypeptide is produced.

As used herein the phrase “gene therapy” refers to the transfer of heterologous genetic information into cells for the therapeutic treatment of diseases or disorders. The heterologous nucleotide sequence is transferred into a cell and is expressed to produce a polypeptide or untranslated RNA molecule.

“Genomic RNA” refers to RNA which contains all of the genetic information required to direct its own amplification or self-replication in vivo, within a target cell. To direct its own replication, the RNA molecule may: 1) encode one or more polymerase, replicase, or other proteins which may interact with viral or host cell-derived proteins, nucleic acids or ribonucleoproteins to catalyze the RNA amplification process; and 2) contain cis RNA sequences required for replication, which may be bound during the process of replication by its self-encoded proteins, or non-self-encoded cell-derived proteins, nucleic acids or ribonucleoproteins, or complexes between any of these components. An alphavirus-derived genomic RNA molecule should contain the following ordered elements: 5′ viral or defective-interfering RNA sequence(s) required in cis for replication, sequences which, when expressed, code for biologically active alphavirus nonstructural proteins (e.g., nsP1, nsP2, nsP3, nsP4), 3′ viral sequences required in cis for replication, and a polyadenylate tract. The alphavirus-derived genomic RNA vector replicon also may contain a viral subgenomic “junction region” promoter which may, in certain embodiments, be modified in order to prevent, increase, or reduce viral transcription of the subgenomic fragment, and sequences which, when expressed, code for biologically active alphavirus structural proteins (e.g., C, E3, E2, 6K, E1). Generally, the term genomic RNA refers to a molecule of positive polarity, or “message” sense, and the genomic RNA may be of length different from that of any known, naturally-occurring alphavirus. In those instances where the genomic RNA is to be packaged into a recombinant alphavirus particle, it must contain one or more sequences which serve to initiate interactions with alphavirus structural proteins that lead to particle formation, and preferably is of a length which is packaged efficiently by the packaging system being employed.

“Alphavirus vector construct” refers to an assembly which is capable of directing the expression of a sequence(s) or gene(s) of interest. Such vector constructs are comprised of a 5′ sequence which is capable of initiating transcription of an alphavirus RNA (also referred to as 5′CSE, in background), as well as sequences which, when expressed, code for biologically active alphavirus nonstructural proteins (e.g., nsP1, nsP2, nsP3, nsP4), and an alphavirus RNA polymerase recognition sequence (also referred to as 3′CSE, in background). In addition, the vector construct may include a viral subgenomic “junction region” promoter which may, in certain embodiments, be modified in order to prevent, increase, or reduce viral transcription of the subgenomic fragment, and also a polyadenylate tract. The vector also may include sequences from one or more structural and/or nonstructural protein genes or portions thereof, extraneous nucleic acid molecule(s) which are of a size sufficient to allow production of viable virus, a 5′ promoter which is capable of initiating the synthesis of viral RNA in vitro from cDNA, a heterologous sequence to be expressed, as well as one or more restriction sites for insertion of heterologous sequences.

“Alphavirus RNA vector replicon”, “RNA vector replicon” and “replicon” refers to a RNA molecule which is capable of directing its own amplification or self-replication in vivo, within a target cell. To direct its own amplification, the RNA molecule may: 1) encode one or more polymerase, replicase, or other proteins which may interact with viral or host cell-derived proteins, nucleic acids or ribonucleoproteins to catalyze RNA amplification; and 2) contain cis RNA sequences required for replication which may be bound by its self-encoded proteins, or non-self-encoded cell-derived proteins, nucleic acids or ribonucleoproteins, or complexes between any of these components. In certain embodiments, the amplification also may occur in vitro. An alphavirus-derived RNA vector replicon molecule should contain the following ordered elements: 5′ viral sequences required in cis for replication (also referred to as 5′ CSE, in background), sequences which, when expressed, code for biologically active alphavirus nonstructural proteins (e.g., nsP1, nsP2, nsP3, nsP4), 3′ viral sequences required in cis for replication (also referred to as 3′ CSE, in background), and a polyadenylate tract. The alphavirus-derived RNA vector replicon also may contain a viral subgenomic “junction region” promoter which may, in certain embodiments, be modified in order to prevent, increase, or reduce viral transcription of the subgenomic fragment, sequences from one or more structural and/or nonstructural protein genes or portions thereof, extraneous nucleic acid molecule(s) which are of a size sufficient to allow production of viable virus, as well as heterologous sequence(s) to be expressed. The source of RNA vector replicons in a cell may be from infection with a virus or recombinant alphavirus particle, or transfection of plasmid DNA or in vitro transcribed RNA.

“Recombinant Alphavirus Particle” refers to a virion unit containing an alphavirus RNA vector replicon. Generally, the recombinant alphavirus particle comprises one or more alphavirus structural and/or nonstructural proteins, a lipid envelope and an RNA vector replicon. Preferably, the recombinant alphavirus particle contains a nucleocapsid structure that is contained within a host cell-derived lipid bilayer, such as a plasma membrane, in which alphaviral-encoded envelope glycoproteins are embedded. The particle may also contain other components (e.g., targeting elements such as biotin, other viral structural and/or nonstructural proteins, or other receptor binding ligands) which direct the tropism of the particle from which the alphavirus was derived, or other RNA molecules.

“Structural protein expression cassette” refers to a nucleic acid molecule which is capable of directing the synthesis of one or more alphavirus structural proteins. The expression cassette should include a 5′ promoter which is capable of initiating the synthesis of RNA from cDNA in vivo, as well as sequences which, when expressed, code for one or more biologically active alphavirus structural proteins (e.g., C, E3, E2, 6K, E1), and a 3′ sequence which controls transcription termination. The expression cassette also may include a 5′ sequence which is capable of initiating transcription of an alphavirus RNA (also referred to as 5′ CSE, in background), a viral subgenomic “junction region” promoter, and an alphavirus RNA polymerase recognition sequence (also referred to as 3′ CSE, in background). In certain embodiments, the expression cassette also may include splice recognition sequences, a catalytic ribozyme processing sequence, a sequence encoding a selectable marker, a nuclear export signal, as well as a polyadenylation sequence. In addition, expression of the alphavirus structural protein(s) may, in certain embodiments, be regulated by the use of an inducible promoter. In addition, the term “structural protein expression cassette” also includes the use of a structural promoter to express foreign genes. For example, some Sindbis vectors use the structural promoter to express foreign genes either by replacing the structural genes or by engineering an additional structural promoter upstream or downstream of the structural genes. Therefore, when the structural protein expression cassette expresses one or more foreign genes, these genes may be expressed in combination with the structural protein(s), or the one or more foreign genes may be substituted for one or more structural proteins.

“Stable Transformation” refers to the introduction of a nucleic acid molecule into a living cell, and long-term or permanent maintenance of that nucleic acid molecule in progeny cells through successive cycles of cell division. The nucleic acid molecule may be maintained in any cellular compartment, including, but not limited to, the nucleus, mitochondria, or cytoplasm. In preferred embodiments, the nucleic acid molecule is maintained in the nucleus. Maintenance may be intrachromosomal (integrated) or extrachromosomal, as an episomal event.

“Alphavirus packaging cell line” refers to a cell which contains a construct as described herein and which produces recombinant alphavirus particles after introduction of an alphavirus vector construct, RNA vector replicon, eukaryotic layered vector initiation system, or recombinant alphavirus particle. The parental cell may be of mammalian or non-mammalian origin. Within preferred embodiments, the packaging cell line is stably transformed with the construct described herein.

“Alphavirus producer cell line” refers to a cell line which is capable of producing recombinant alphavirus particles, comprising an alphavirus packaging cell line which also contains an alphavirus vector construct, RNA vector replicon, eukaryotic layered vector initiation system, or recombinant alphavirus particle. Preferably, the alphavirus vector construct is eukaryotic layered vector initiation system, and the producer cell line is stably transformed with the vector construct. In preferred embodiments, transcription of the alphavirus vector construct and subsequent production of recombinant alphavirus particles occurs only in response to one or more factors, or the differentiation state of the alphavirus producer cell line.

“Gene delivery vehicle” refers to a construct which can be utilized to deliver a gene or sequence of interest. Representative examples include alphavirus RNA vector replicons, alphavirus vector constructs, eukaryotic layered vector initiation systems and recombinant alphavirus particles.

“Treatment” refers to both therapeutic treatment and prophylactic or preventative measures. Those in need of treatment include those already with the disorder as well as those in which the disorder is to be prevented.

A “disorder” is any condition that would benefit from treatment with the polypeptide. This includes chronic and acute disorders or diseases including those pathological conditions which predispose the mammal to the disorder in question.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hopatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

When the terms “one,” “a,” or “an” are used in this disclosure, they mean “at least one” or “one or more,” unless otherwise indicated.

Numerous aspects and advantages of the invention will be apparent to those skilled in the art upon consideration of the following detailed description which provides illumination of the practice of the invention.

Alphaviruses express four nonstructural proteins, designated nsp1, nsp2, nsp3, and nsp4. Vectors of the present invention derived from alphaviruses should contain sequences encoding the four nonstructural proteins. In wild-type Sindbis virus, nonstructural proteins 1-3 are encoded by nucleotides 60 to 5747, while nsP4 is encoded by nucleotides 5769 to 7598. The nucleotide sequences for nsp1, nsp2, nsp3 and nsp4 have been assigned SEQ ID NOS:1, 3, 5, and 7, respectively. The amino acid sequences for nsP1, nsP2, nsP3 and nsP4 have been assigned SEQ ID NOs:2, 4, 6, and 8, respectively. The nonstructural proteins are translated from the genomic positive strand RNA as one of two large polyproteins, known as P123 or P1234, respectively, depending upon (i) whether there is an opal termination codon between the coding regions of nsP3 and nsP4 and (ii) if there is such an opal codon present, whether there is translation termination of the nascent polypeptide at that point or readthrough and hence production of P1234. The opal termination codon is present at the nsP3/nsP4 junction of the alphaviruses SIN (strain AR339 and the SIN-1 strain described herein), AURA, WEE, EEE, VEE, and RR, and thus the P123 and P1234 species are expressed in cells infected with these viruses. In contrast, no termination codon is present at the nsP3/nsP4 junction of the alphaviruses SIN (strain AR86, SF, and ONN), and thus only the P1234 species is expressed in cells infected with these viruses. Both the polyprotein and processed monomeric forms of the nonstructural proteins function in the replication of the alphavirus RNA genome. Experiments examining growth characteristics of alphavirus nonstructural protein cleavage mutants have indicated that the polyproteins are involved in the synthesis of the genomic negative stranded RNA, while the individual monomeric proteins catalyze the synthesis of the genomic and subgenomic positive stranded RNA species (Shirako and Strauss, J. Virol. 68:1874-1885, 1974). Translational readthrough generally occurs about 10%-20% of the time in cells infected with wild type Sindbis virus containing the opal termination codon at the nsP3/nsP4 junction. Processing of P123 and P1234 is by a proteinase activity encoded by the one of the nonstructural proteins, and is discussed further below. The order of processing, whether in cis or in trans, depends on various factors, including the stage of infection. For example, Sindbis virus and SFV produce P123 and nsp4 early in infection, and P12 and P34 later in infection. Further processing then releases the individual nonstructural proteins. Each nonstructural protein has several functions, some of which are described below.

Nonstructural protein 1 is required for the initiation of (or continuation of) minus-strand RNA synthesis. It also plays a role in capping the 5′ terminus of genomic and subgenomic alphavirus RNAs during transcription, as nsP1 possesses both methyltransferase (Mi and Stollar, Vir. 184:423-427, 1991) and guanyltransferase activity (Strauss and Strauss, Microbiol. Rev. 58(3):491-562. 1994). NsP1 also modulates the proteinase activity of nsP2, as polyproteins containing nsP1 inefficiently cleave between nsP2 and nsP3 (de Groot et al., EMBO J. 9:2631-2638, 1990).

Nonstructural protein 2 is a multifunctional protein, involved in the replication of the viral RNA and processing of the nonstructural polyprotein. The N-terminal domain of the protein (spanning about the first 460 amino acids) is believed to be a helicase which is active in duplex unwinding during RNA replication and transcription. Synthesis of 26S subgenomic mRNA, which, in vectors according to the present invention, encodes the gene(s) of interest, requires functional nsP2. The C-terminal domain of nsP2, between amino acid residues 460-807 of Sindbis virus, proteolytically cleaves in trans and in cis the nonstructural polyprotein between the nsP1/nsP2, nsP2/nsP3, and nsP3/nsP4 junctions. Alignment of the primary sequences of the alphavirus nsP2 C-terminal domains suggests that nsP2 is a papain-like proteinase (Hardy and Strauss, J. Virol. 63:4653-4664, 1988).

Other observed characteristics of nsP2 have not, as yet, been assigned a function directly related to the propagation of alphaviruses. For example, it has been shown that nsP2 is closely associated with ribosomes in SFV-infected cells, and can be cross-linked to rRNA by UV irradiation (Ranki et al., FEBS Lett. 108:299-302, 1979). Further, 50% of nsP2 is localized in the nuclear matrix, particularly in the area of the nucleoli of SFV-infected BHK cells (Peranen et al., J. Virol. 64:1888-1896, 1990). Localization of nsP2 to the nuclei presumably proceeds by active transport, as it exceeds the size of small proteins and metabolites (about 20-60 kD), which can enter the nucleus by diffusion through nuclear core complexes (Paine et al., Nature 254:109-114, 1975). Putative NLS sequences have been identified in the alphaviruses SFV, SIN, RR, ONN, OCK, and VEE (Rikkonen et al., Vir. 189:462-473, 1992).

Nonstructural protein nsP3 contains two distinct domains, although their precise roles in viral replication are not well understood. The N-terminal domain ranges in length from 322 to 329 residues in different alphaviruses and exhibits a minimum of 51% amino acid sequence identity among any two alphaviruses. The C-terminal domain, however, is not conserved among known alphaviruses in length or in sequence, and multiple changes are tolerated (Li et al., Virology, 179:416-427). The protein is found associated with replication complexes in a heavily phosphorylated state. In alphaviruses whose genomes contain an opal termination codon between the nsP3/nsP4 junctions, two different proteins are produced depending upon whether or not there is readthrough of the opal termination signal. Readthrough results in an nsP3 protein which contains 7 additional carboxy terminal amino acids after cleavage of the polyprotein. It is clear that nsP3 is required in some capacity for viral RNA synthesis, as particular mutants of this protein are RNA negative, and the P123 polyprotein is required for minus-strand RNA synthesis.

NsP4 is the virus-encoded RNA polymerase and contains the GDD motif characteristic of such enzymes (Kamer and Argos, Nucleic Acids Res. 12:7269-7282, 1984). Thus, nsP4 is indispensable for alphavirus RNA replication. The concentration of nsP4 is tightly regulated in infected cells. In most alphaviruses, translation of nsP4 requires readthrough of an opal codon between the nsP3 and nsP4 coding regions, resulting in lower intracellular levels as compared to other nonstructural proteins. Additionally, the bulk of nsP4 is metabolically unstable, through degradation by the N-end rule pathway (Gonda et al., J. Biol. Chem. 264:16700-16712, 1989). However, some nsP4 is stable, due to its association with replication complexes which conceal degradation signals. Thus, stabilization of the enzyme by altering the amino terminal residue may prove useful in promoting more long term expression of proteins encoded by the vectors described herein. Stabilizing amino terminal residues include methionine, alanine, and tyrosine.

The present invention is related to the use of alphaviruses, such as but not limited to, Sindbis virus, as oncolytic agents against cancer cells with constitutively activated signaling pathways. Sindbis virus is generally avirulent to humans and thus does not cause any diseases in humans. As a result, Sindbis virus has often been used as a research tool to produce recombinant proteins in tissue cultures. It has recently been shown that Sindbis virus possess oncolytic activity; however, the present invention demonstrates that Sindbis virus only infects certain types of cancer cells with constitutively activated signaling pathways, such as but not limited to, the Ras signaling pathway. Examples of cancers that may treated as disclosed herein include, but are not limited to, any cancer having a constitutively activated Ras signaling pathway, such as but not limited to, pancreatic cancer, colon cancer, lung cancer, and acute leukemia.

There is general interest in therapeutic viruses, and several viruses have been developed into expression vectors to produce desired proteins in gene therapy. Two viruses have been reported to kill cancer cells directly through oncolytic activity: an adenovirus mutant (a DNA virus) and a reovirus (a double-stranded RNA virus). However, no virus has been developed into a cancer drug at present. Sindbis virus has the advantage of being a small plus-stranded RNA virus that has a simpler genome, a faster growth cycle in the cytoplasm (versus complications in the nuclei), is easier to be manipulated to produce therapeutic viruses, and kills cancer cells more efficiently within hours. The present invention demonstrates that Sindbis virus specifically targets and kills cancer cells while leaving normal cells untouched. This specificity provides a tremendous advantage over small molecule drugs that are relatively nonspecific and target cancer cells less efficiently.

In another embodiment, the present invention is related to alphavirus vectors, and in particular, but not by way of limitation, to Sindbis viral vectors that encode a fusion of nonstructural protein 3 (nsP3) with a protein/peptide of interest, such as but not limited to, a marker, diagnostic or therapeutic protein/peptide. The recombinant functional fusion protein comprising nsP3 and the protein of interest is produced without affecting virus replication or infection. In one embodiment, the coding sequence for the protein/peptide of interest is inserted into the C-terminal domain of the nsP3 coding sequence, as the C-terminal domain of the nsP3 protein is not as conserved between alphaviruses (as compared to the N-terminal domain) and is thus more acceptable to manipulation.

Examples of diagnostic proteins/peptides that may be utilized in accordance with the present invention include any marker protein/peptide that is expressible in the form of a fusion protein and which can be utilized to follow or trace the viral vectors during infection. Specific examples include, but are not limited to, fluorescent proteins such as GFP, RFP, CFP, YFP, and the like; antibiotic resistance markers ampicillin, kanamycin, tetracycline, and the like; enzyme markers such as beta-galactosidase, chloramphenicol transferase (CAT), luciferase, and the like.

Examples of therapeutic proteins/peptide that may be utilized in accordance with the present invention include any therapeutic protein/peptide that is expressible in the form of a fusion protein and that can function to directly kill a certain cell type (such as but not limited to, a cancer cell) or function to trigger an immune response. Specific examples include, but are not limited to, interferons, cytokines such as IL-2, tumor antigens such as human papillomavirus tumor antigen E7 and other specific antigens from influenza virus, herpes simplex virus, hantavirus, malaria parasite, and any other tumor antigens known in the art.

The present invention overcomes the disadvantages and defects of the prior art in that it provides the ability for foreign proteins to be functionally expressed in the nonstructural region of the virus, whereas the prior art vectors were limited to expression in the structural regions of the virus.

Insertion/deletion studies on the nsP3 region of the Sindbis virus have been conducted, and are described in detail in LaStarza et al. (J. Virol. 68:5781-5791 (1994)), which is attached hereto and incorporated herein by reference.

The present invention further includes the combination of the recombinant alphavirus vectors encoding nsP3 and protein/peptide of interest fusion with one or more of the prior art vectors having a fusion in a structural region thereof, thus yielding a single recombinant alphavirus vector that can express two foreign genes simultaneously (one foreign gene expressed in a structural region thereof and the second expressed in a nonstructural region thereof. In this manner, the vector may express two marker proteins/peptides, two therapeutic proteins/peptides, or a combination of markers protein/peptide and one therapeutic protein/peptide.

Any alphavirus vector described herein or known in the art may be utilized as the second vector having a fusion in a structural region thereof. Examples of vectors that may be utilized include, but are not limited to, any of the vectors described in U.S. Pat. Nos. 7,005,275, issued to Renner et al., on Feb. 28, 2006; 6,635,476 issued to Murphy on Oct. 21, 2003; 6,465,634, issued to Dubensky et al., on Oct. 15, 2002; 6,376,236, issued to Dubensky et al., on Apr. 23, 2002; 6,239,201, issued to Polo et al., on Dec. 11, 2001; 5,843,723, issued to Dubensky et al., on Dec. 1, 1998; 5,789,245, issued to Dubensky et al., on Aug. 4, 1998; and 5,091,309, issued to Schlesinger et al., on Feb. 25, 1992; the contents of each of which have previously been incorporated herein by reference.

Examples are provided hereinbelow. However, the present invention is to be understood to not be limited in its application to the specific experimentation, results and laboratory procedures. Rather, the Examples are simply provided as one of various embodiments and are meant to be exemplary, not exhaustive.

### EXAMPLE 1

Sindbis virus (SINV) is an enveloped plus-strand RNA virus belonging to the Alphavirus genus of the family Togaviridae. The SINV RNA genome contains 11,703 nucleotides and is completely sequenced. With increasing understanding of the viral genome organization and replication strategy, SINV has been developed into one of the most efficient expression vectors for gene transfer in cultured cell lines and animal models. SINV vectors have been used to deliver genes to antigen-presenting cells to trigger specific immune responses to pathogens and cancers. Furthermore, recent studies show that SINV vectors are targeted to and exhibit oncolytic activity towards a variety of tumors in vivo, making them promising gene therapy vehicles against cancer.

There are generally two types of SINV vectors: replicons and infectious recombinant viruses. The replicons contain only viral nonstructural genes whose products (nsP1-4) are responsible for viral RNA replication/amplification, while viral structural genes (capsid and envelope glycoproteins) are replaced by foreign genes to be expressed. Because of the lack of structural proteins, the replicons are not packaged into infectious virions and thus are self-contained in the transfected cells. However, they can form infectious virions if the structural proteins are provided in trans. The more recently developed DNA vectors can perhaps be viewed as a form of replicons. The recombinant viruses, on the other hand, contain all SINV genetic information with foreign genes inserted in certain structural regions of the viral genome under the control of a SINV subgenomic promoter. These are live viruses that undergo normal viral replication cycle and in the process express the cloned foreign genes.

All SINV vectors so far keep the nonstructural region intact, because it encodes the nonstructural proteins (nsP1-4) for the amplification of viral RNA, which in turn is important for the expression of foreign genes. Several point mutations in the nsP2 gene are found to reduce the viral RNA replication to a relatively low level that no longer causes cytopathic effect and readily establishes persistent infection. These mutations have been incorporated into a new class of non-cytopathic SINV vectors. In the current study, the possibility of direct cloning and expression of foreign genes in the nonstructural region was explored. To this end, a recombinant SINV that contains an in-frame insertion of the entire cDNA of GFP (green fluorescent protein) in nsP3 was generated, which is known to tolerate some in-frame insertions and deletions in its C-terminal variable domain. This recombinant SINV (termed Toto1101/GFP) remains infectious and grows to the same high titer as the parental virus, demonstrating the plasticity of nsP3 and the feasibility of expressing a functional fusion protein in this region. Incorporation of this type of modification into SINV vectors should improve their utility in terms of simultaneous expression of multiple genes in both structural and nonstructural regions.

II. Materials and Methods

Cells: The BHK-21 (baby hamster kidney) cell line was used in this study. Cell monolayers were grown in 35-mm tissue culture dishes or 6-well plates in a -MEM (invitrogen) containing 5% fetal bovine serum (FBS) (Invitrogen) and incubated at 37° C. in a cell culture incubator with 5% CO₂.

Generation of the Toto1101/GFP virus: The GFP cDNA was amplified by PCR with pEGFP-C1 (BD Biosciences) as template and oligonucleotide primers containing SpeI site. After digestion with SpeI, the GFP cDNA was directly inserted into the unique SpeI site of Toto1101, a full-length cDNA clone of Sindbis virus. The nsp3/GFP nucleotide sequence has been assigned SEQ ID NO:9, while the amino acid sequence has been assigned SEQ ID NO:10. The Toto1101/GFP nucleotide sequence has been assigned SEQ ID NO:11. In the resulting Toto1101/GFP plasmid, the GFP reading frame was the same as that of nsP3, and the translation product was expected to be a nsP3-GFP fusion protein with GFP sandwiched between amino acids 388 and 389 of nsP3. The GFP cDNA sequence was confirmed by direct DNA sequencing. The Toto1101/GFP plasmid was then used as a template for in vitro transcription by SP6 RNA polymerase as previously described and the resulting RNA transcript was used for transfection of BHK-21 cell monolayers via lipofectin-mediated procedure (Invitrogen). Briefly, 1 μg of the RNA transcript was mixed with 200 μl of PBS (phosphate-buffered saline) containing 8 μg of lipofectin and incubated on ice for 10 min. The transfection mixture was then added to the cell monolayers and incubated at room temperature for 10 min, before replacing the transfection mixture with 3 ml of growth medium (α-MEM containing 5% FBS). The cells were incubated at 37° C. in an incubator with 5% CO₂. The medium containing the released viruses was harvested 48 h later and frozen at −80° C. as virus stocks.

Titration of virus stocks by plaque assay: Virus stocks were serially diluted in PBS containing 1% FBS and 200 μl of each dilution were added to each well of BHK-21 cell monolayers. The cells were incubated at room temperature for 30 min, followed by direct overlay of each well with 3 ml of 1% agarose in α-MEM. The cells were then incubated at 37° C. in an incubator with 5% CO₂. After 3 days, the cells were fixed by adding 1 ml of 7% formaldehyde to each well and incubating at room temperature for 30 min. The agarose overlay was then carefully removed with a spatula and cell monolayers were stained with crystal violet to visualize and count the number of viral plaques.

One-step growth curve: BHK-21 cell monolayers in 35-mm culture dishes were infected with Toto1101/GFP and the parental Toto1101 viruses, diluted in 200 μl of PBS containing 1% FBS, at a multiplicity of infection (MOI) of 20 PFU/cell. After virus absorption at room temperature for 1 h, the infection mix was aspirated and the cells were rinsed with PBS, followed by addition of 3 ml α-MEM containing 5% FBS to each dish and incubation in a 37° C. incubator with 5% CO₂. At 1 h post-infection, the medium was replaced with 3 ml of fresh pre-warmed (37° C.) α-MEM containing 5% FBS, followed by continued incubation at 37° C. A small amount of medium (10 μl) was collected from each dish at the following time points: 4 h, 6 h, 8 h, 12 h and 24 h post-infection and the virus titer in the medium was determined by the plaque assay as described above.

Pulse-chase labeling and immunoprecipitation of nsP3: BHK-21 cell monolayers in 35-mm culture dishes were infected with Toto1101/GFP and the parental Toto1101 viruses as described above. At 3 h post-infection, the medium was removed and the cells were pulse-labeled for 15 min in methionine-free α-MEM containing 50 Ci/ml ³⁵S-methionine (MP Biomedicals), followed by different times of chase as indicated in normal growth medium (α-MEM with 5% FBS). The cells were then rinsed once with ice-cold PBS and lysed with 200 μl of 1% sodium dodecyl sulfate (SDS). The lysates were boiled for 3 min and either stored at −70° C. or used directly for immunoprecipitation with the rabbit antiserum monospecific for nsP3 as previously described. The immunoprecipitated proteins on γBind G-Sepharose beads (Amersham Biosciences) were resuspended in 30 μl of Laemmli sample buffer (50 mM Tris-HCl, pH 6.8, 1% SDS, 1% β-mercaptoethanol, 12.5% glycerol, 0.01% bromophenol blue), boiled for 3 min and centrifuged for 1 min. Proteins released in the supernatants were separated by SDS-polyacrylamide gel electrophoresis (PAGE). The gels were treated with 1 M sodium salicylate (in 10% methanol) for 30 min, dried and exposed to Kodak BioMax films for autoradiography.

Confocal fluorescence microscopy: BHK-21 cells were grown on cover slips and infected with the Toto1101/GFP virus as described above. At the indicated times, cells were rinsed once with PBS and fixed in 4% paraformaldehyde (in PBS) (Electron Microscopy Sciences) for 30 min. The cover slips were mounted in PBS on glass slides (Fisher Scientific) and the fluorescence was observed and documented via a Leica confocal laser scanning microscope (Liang and Li, 2000).

Results and Discussion

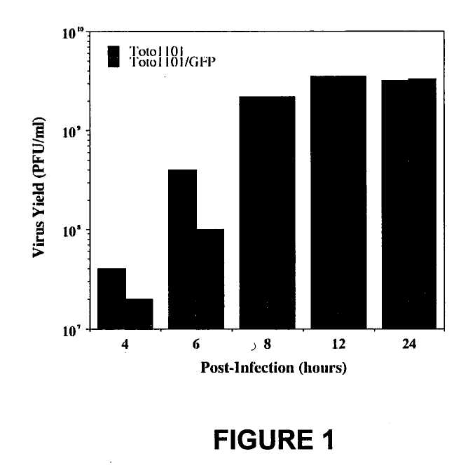

First, it was demonstrated that the Toto1101/GFP virus is viable and grows to high titer. The Toto1101/GFP RNA transcript produced the same high titer of infectious virions (2×10⁹ PFU/ml) as the parental Toto1101 transcript, indicating that nsP3 remains functional, despite the GFP insertion. The single-step growth curve of the viruses at 37° C. was then determined. The Toto1101/GFP and Toto1101 virus stocks derived from the transfection were used to infect fresh BHK cell monolayers at 20 PFU/cell, and media containing released viruses were collected at the indicated times after infection, followed by titration of the virus samples. The Toto1101/GFP virus replication showed a small but reproducible delay early in infection, with lower virus yields (2-4 fold) than the parental Toto1101 virus at 4 h and 6 h post-infection (FIG. 1). However, Toto1101/GFP quickly caught up, and the virus yield was essentially the same as that of Toto1101 at 8 h post-infection and throughout later time points (FIG. 1).

Next, the infectivity of the RNA transcript of Toto1101/GFP was examined directly in comparison to that of parental Toto1101 by plaque assays upon transfection. Consistent with the delay in the growth curve (FIG. 1), Toto1101/GFP RNA transcript produced somewhat less plaques (5×10⁵ PFU/m g) than the parental Toto1101 transcript (1.5×10⁶ PFU/m g), reflecting some negative effect on nsP3, which is known to function in viral RNA synthesis. However, the plaque morphology and virus yields are the same for both viruses.

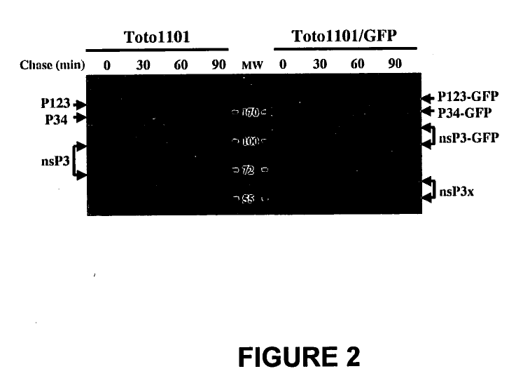

Next, it was demonstrated that the nsP3-GFP fusion protein is expressed properly and post-translationally processed. Pulse-chase labeling experiments were performed with [³⁵S]methionine (ICN Translabel) in Toto1101/GFP and Toto1101 virus-infected cells, followed by immunoprecipitation with a nsP3-specific rabbit antiserum, SDS-PAGE and autoradiography. It was previously reported that after a short labeling (10-15 min), newly made nsP3 appeared as a 70 kDa protein. However, it was gradually chased into multiple phosphorylated forms with the largest, predominant form migrating at approximately 100 kDa. This observation was confirmed with the parental Toto1101 virus, which served as a control (FIG. 2). In this case, immediately after the 15-min pulse-labeling, polyprotein precursors P123 and P34 were observed in addition to the processed nsP3 (FIG. 2, indicated on the left). Most of P123 was chased into nsP3 within 30 min, but P34 was stable throughout the chase (as long as 90 min). The mature nsP3 product was chased into higher molecular weight phosphorylated forms ranging from 72-100 kDa (FIG. 2). In Toto1101/GFP-infected cells, on the other hand, the 15-min pulse-labeling produced the corresponding precursors P123-GFP and P34-GFP as well as the processed mature nsP3-GFP, which is larger (about 100 kDa) than nsP3 itself reflecting GFP insertion (FIG. 2, indicated on the right). Both the expected size and the recognition by the nsP3-specific antibody indicated that this 100 kDa protein is the nsP3-GFP fusion protein. Like their Toto1101 counterparts, P123-GFP was mostly chased into nsP3-GFP within 30 min with a slightly slower kinetics, while P34-GFP was stable throughout the 90-min chase (FIG. 2, indicated on the right). Likewise, nsP3-GFP was chased into higher molecular weight forms, demonstrating proper post-translational processing (phosphorylation) of the protein (FIG. 2). It was noticed that a new nsP3-related fragment, which was termed nsP3x (FIG. 2), appeared only in Toto1101/GFP-infected cells. It was smaller than nsP3, but was recognized by nsP3 antibody and was chased into a higher molecular weight form (FIG. 2). It was likely a degradation product of nsP3-GFP.

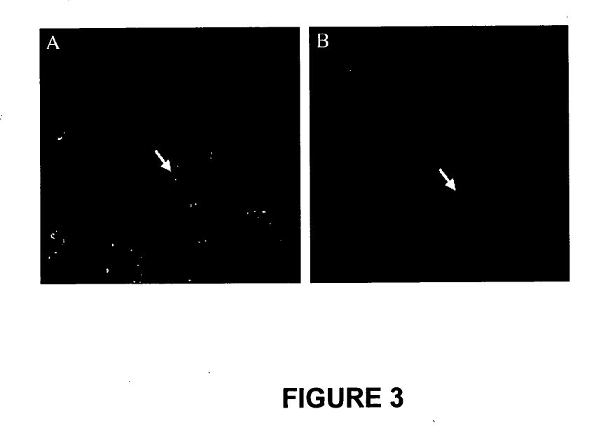

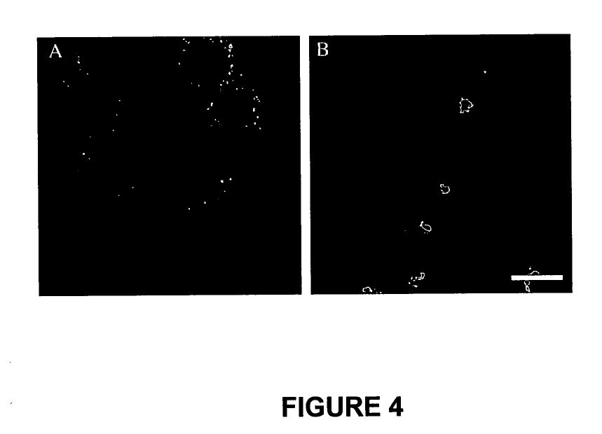

Then, the localization of nsP3-GFP to the plasma membrane and intracellular structures was demonstrated. Proper folding and functionality of the GFP portion of the fusion protein was determined by examining the green fluorescence of GFP via confocal fluorescence microscopy during the time course of Toto1101/GFP virus infection of BHK cells. This also allowed the determination of the intracellular localization of nsP3-GFP. The GFP fluorescence was detected as early as 2 h post-infection (FIG. 3). At this time, nsP3-GFP was found at the plasma membrane (FIG. 3, indicated by arrows) as well as in punctuate intracellular structures. Interestingly, the plasma membrane-localized nsP3-GFP tended to be at the junction region between neighboring cells rather than at the free rim of the cell (FIG. 3B). By 3 h post-infection, however, all nsP3-GFP moved to the punctuate intracellular structures (FIG. 4A) and remained there throughout virus infection. These nsP3-GFP containing intracellular structures moved progressively towards the center of the cell during the course of infection and eventually clustered at the perinuclear region at late infection (10 h post-infection) (FIG. 4B). The punctuate pattern of the nsP3-GFP structures is similar to the nsP3-positive late endosome/lysosome-like vesicles in SINV-infected cells described previously.

The early targeting of nsP3-GFP, probably in the form of polyproteins P1234 and P123, to the plasma membrane suggests that functional viral RNA replication/transcription complexes may form and initiate the minus-strand RNA synthesis at the plasma membrane before moving inward to the cytoplasm, possibly via endocytosis. Further studies may provide insight into the functional differences between plasma membrane-localized and late endosome/lysosome-localized viral RNA replication/transcription complexes and our Toto1101/GFP virus should be useful in monitoring the formation and dynamics of the replication/transcription complexes in live cells. In addition, functional expression of GFP in nsP3 demonstrates that other reporter or therapeutic genes may also be expressed in this nonstructural region, thus improving the utility of SINV expression vectors. Because the nonstructural genes are usually expressed at a lower level than the structural genes, foreign genes cloned in this region are likely to express at a lower level as well.

### EXAMPLE 2

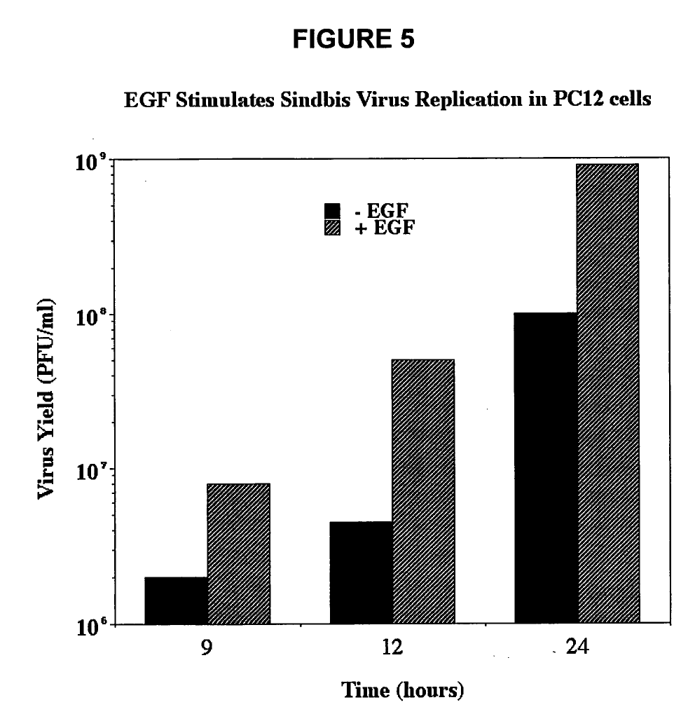

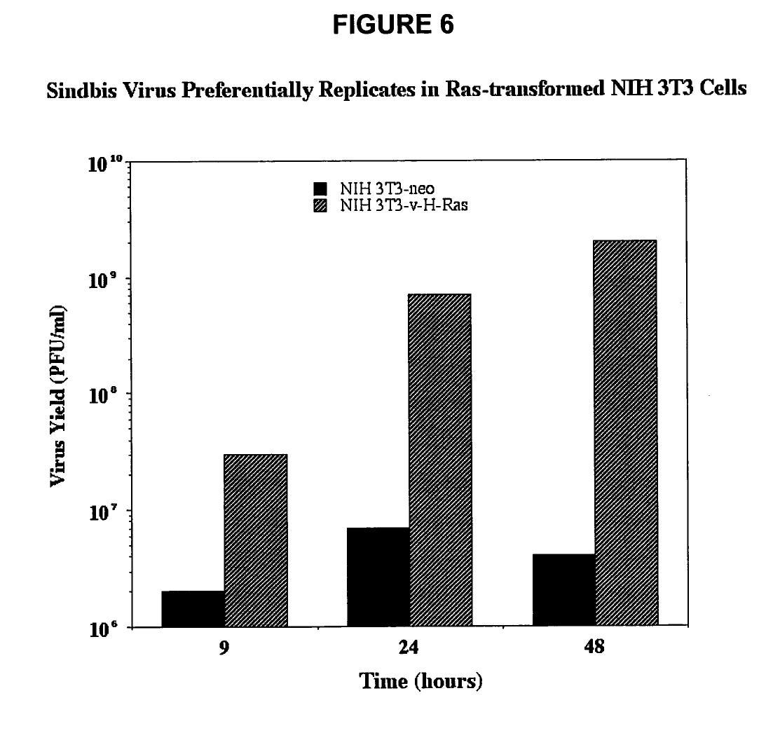

With the Toto1000 plasmid containing the full-length cDNA of Sindbis virus, it was possible to make Sindbis virus stocks through in vitro transcription and transfection of cultured baby hamster kidney (BHK) cells. The virus growth curves were then determined in cultured PC12 and HeLa cells in the presence and absence of epidermal growth factor (EGF) (see FIG. 5). It was found that Sindbis virus grows up to an order of magnitude faster in the EGF-treated cells. It is known that EGF stimulates cell growth via activating the Ras signaling pathway; therefore, the virus growth curve was determined in two NIH 3T3 cell lines: one cell line that expresses v-H-Ras (a constitutively activated form of Ras) and a control cell line that was transfected with an empty vector and therefore does not express the v-H-Ras protein. FIG. 6 illustrates that Sindbis virus only infects and kills the cells expressing v-H-Ras but not the control (normal) cells. It is well known that v-H-Ras causes cancer, and in fact, the Ras gene is the most frequently mutated/activated oncogene with about 30% of all cancers containing Ras mutations that constitutively activate Ras function in cell growth. It is believed that Sindbis virus and related plus-stranded RNA viruses have great potential as oncolytic reagents to specifically kill cancer cells in cancer therapy.

Materials and Methods: PC12 cells were grown in 35-mm tissue culture dishes with DMEM containing 10% horse serum and 5% fetal bovine serum (Invitrogen), while Hela and NIH3T3 cells (both v-H-ras transformed and control cell lines) were grown in DMEM containing 5% fetal bovine serum. PC12 and Hela cells were either treated or not treated with EGF (50 ng/ml, BD Biosciences) for 12 hours. The Toto1101/GFP virus was generated as described in Example 1 and used for infection of the cells, followed by collection of viral samples at different times as indicated in FIGS. 5 and 6, and the virus titers and one-step growth curves were determined by plaque assays as described in Example 1.

Thus, in accordance with the present invention, there has been provided recombinant alphavirus vectors, as well as methods of making and using same, that fully satisfies the objectives and advantages set forth hereinabove. Although the invention has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the invention.

What is claimed is:

1. A purified nucleic acid segment, wherein the purified nucleic acid segment comprises:
a nucleic acid segment encoding a fusion protein comprising a Sindbis virus nonstructural protein and a protein or peptide of interest.

2. The purified nucleic acid segment, wherein the Sindbis virus nonstructural protein is nonstructural protein 3.

3. The purified nucleic acid segment of claim 1, wherein the protein or peptide of interest is selected from the group consisting of a fluorescent protein, an antibiotic resistance marker, an enzyme marker, a cytotoxic agent, an interferon, a cytokine, a tumor antigen a viral antigen, and combinations thereof.

4. The purified nucleic acid segment of claim 1, wherein the portion of the purified nucleic acid segment encoding the protein or peptide of interest is inserted into a portion of the purified nucleic acid segment encoding the C-terminal domain of the nonstructural protein.

5. A recombinant alphavirus vector, comprising:
a Sindbis viral vector comprising a nucleic acid segment encoding a fusion protein comprising a Sindbis virus nonstructural protein and a protein or peptide of interest; and
wherein the production of the fusion protein does not affect viral replication or infection.

6. The recombinant alphavirus vector of claim 5, wherein the Sindbis virus nonstructural protein is nonstructural protein 3.

7. The recombinant alphavirus vector of claim 5, wherein the protein or peptide of interest is selected from the group consisting of a fluorescent protein, an antibiotic resistance marker, an enzyme marker, a cytotoxic agent, an interferon, a cytokine, a tumor antigen a viral antigen, and combinations thereof.

8. The recombinant alphavirus vector of claim 5, wherein the portion of the purified nucleic acid segment encoding the protein or peptide of interest is inserted into a portion of the purified nucleic acid segment encoding the C-terminal domain of the nonstructural protein.

9. The recombinant alphavirus vector of claim 5, wherein the Sindbis viral vector further comprises a structural protein expression cassette.

10. A recombinant alphavirus particle, comprising:
a virion unit comprising a recombinant alphavirus vector, comprising:
a Sindbis viral vector comprising a nucleic acid segment encoding a fusion protein comprising a Sindbis virus nonstructural protein and a protein or peptide of interest; and
wherein the production of the fusion protein does not affect viral replication or infection.

11. The recombinant alphavirus particle of claim 10, wherein the Sindbis virus nonstructural protein is nonstructural protein 3.

12. The recombinant alphavirus particle of claim 10, wherein the protein or peptide of interest is selected from the group consisting of a fluorescent protein, an antibiotic resistance marker, an enzyme marker, a cytotoxic agent, an interferon, a cytokine, a tumor antigen a viral antigen, and combinations thereof.

13. The recombinant alphavirus particle of claim 10, wherein the portion of the purified nucleic acid segment encoding the protein or peptide of interest is inserted into a portion of the purified nucleic acid segment encoding the C-terminal domain of the nonstructural protein.

14. The recombinant alphavirus particle of claim 10, wherein the Sindbis viral vector further comprises a structural protein expression cassette.

15. A method of specifically killing tumor cells, comprising the steps of:
providing recombinant alphavirus particles, wherein each recombinant alphavirus particle comprises:
a virion unit comprising a recombinant alphavirus vector, comprising:
a Sindbis viral vector comprising a nucleic acid segment encoding a fusion protein comprising a Sindbis virus nonstructural protein and a protein or peptide of interest; and
wherein the production of the fusion protein does not affect viral replication or infection; and
infecting the tumor cells with the recombinant alphavirus particles, whereby the recombinant alphavirus particles specifically kill the tumor cells.

16. The method of claim 15, wherein the recombinant alphavirus particles do not kill normal, non-cancerous cells.

17. The method of claim 15, wherein the tumor cells have a constitutively activated Ras signaling pathway.

18. The method of claim 15 wherein, in the step of providing the recombinant alphavirus particle, the Sindbis virus nonstructural protein is nonstructural protein 3.

19. The method of claim 15 wherein, in the step of providing the recombinant alphavirus particle, the protein or peptide of interest is selected from the group consisting of a fluorescent protein, an antibiotic resistance marker, an enzyme marker, a cytotoxic agent, an interferon, a cytokine, a tumor antigen a viral antigen, and combinations thereof.

20. The method of claim 15 wherein, in the step of providing the recombinant alphavirus particle, the portion of the purified nucleic acid segment encoding the protein or peptide of interest is inserted into a portion of the purified nucleic acid segment encoding the C-terminal domain of the nonstructural protein.

21. The method of claim 15 wherein, in the step of providing the recombinant alphavirus particle, the Sindbis viral vector further comprises a structural protein expression cassette.


SEQUENCE LISTING (11 sequences)

SEQ ID NO: 1 - DNA, 1620 bp, Sindbis virus
atggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagca

SEQ ID NO: 2 - protein, 540 aa, Sindbis virus
MEKPVVNVDVDPQSPFVVQLQKSFPQFEVVAQQVTPNDHANARAFSHLASKLIELEVPTTATILDIGSAPARRMFSEHQYHCVCPMRSPEDPDRMMKYASKLAEKACKITNKNLHEKIKDLRTVLDTPDAETPSLCFHNDVTCNMRAEYSVMQDVYINAPGTIYHQAMKGVRTLYWIGFDTTQFMFSAMAGSYPAYNTNWADEKVLEARNIGLCSTKLSEGRTGKLSIMRKKELKPGSRVYFSVGSTLYPEHRASLQSWHLPSVFHLNGKQSYTCRCDTVVSCEGYVVKKITISPGITGETVGYAVTHNSEGFLLCKVTDTVKGERVSFPVCTYIPATICDQMTGIMATDISPDDAQKLLVGLNQRIVINGRTNRNTNTMQNYLLPIIAQGFSKWAKERKDDLDNEKMLGTRERKLTYGCLWAFRTKKVHSFYRPPGTQTCVKVPASFSAFPMSSVWTTSLPMSLRQKLKLALQPKKEEKLLQVSEELVMEAKAAFEDAQEEARAEKLREALPPLVADKGIEAAAEVVCEVEGLQADIGA

SEQ ID NO: 3 - DNA, 2421 bp, Sindbis virus
gcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagcc

SEQ ID NO: 4 - protein, 807 aa, Sindbis virus
ALVETPRGHVRIIPQANDRMIGQYIVVSPNSVLKNAKLAPAHPLADQVKIITHSGRSGRYAVEPYDAKVLMPAGGAVPWPEFLALSESATLVYNEREFVNRKLYHIAMHGPAKNTEEEQYKVTKAELAETEYVFDVDKKRCVKKEEASGLVLSGELTNPPYHELALEGLKTRPAVPYKVETIGVIGTPGSGKSAIIKSTVTARDLVTSGKKENCREIEADVLRLRGMQITSKTVDSVMLNGCHKAVEVLYVDEAFACHAGALLALIAIVRPRKKVVLCGDPMQCGFFNMMQLKVHFNHPEKDICTKTFYKYISRRCTQPVTAIVSTLHYDGKMKTTNPCKKNIEIDITGATKPKPGDIILTCFRGWVKQLQIDYPGHEVMTAAASQGLTRKGVYAVRQKVNENPLYAITSEHVNVLLTRTEDRLVWKTLQGDPWIKQPTNIPKGNFQATIEDWEAEHKGIIAAINSPTPRANPFSCKTNVCWAKALEPILATAGIVLTGCQWSELFPQFADDKPHSAIYALDVICIKFFGMDLTSGLFSKQSIPLTYHPADSARPVAHWDNSPGTRKYGYDHAIAAELSRRFPVFQLAGKGTQLDLQTGRTRVISAQHNLVPVNRNLPHALVPEYKEKQPGPVKKFLNQFKHHSVLVVSEEKIEAPRKRIEWIAPIGIAGADKNYNLAFGFPPQARYDLVFINIGTKYRNHHFQQCEDHAATLKTLSRSALNCLNPGGTLVVKSYGYADRNSEDVVTALARKFVRVSAARPDCVSSNTEMYLIFRQLDNSRTRQFTPHHLNCVISSVYEGTRDGVGA

SEQ ID NO: 5 - DNA, 1668 bp, Sindbis virus
gcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtggg

SEQ ID NO: 6 - protein, 556 aa, Sindbis virus
APSYRTKRENIADCQEEAVVNAANPLGRPGEGVCRAIYKRWPTSFTDSATETGTARMTVCLGKKVIHAVGPDFRKHPEAEALKLLQNAYHAVADLVNEHNIKSVAIPLLSTGIYAAGKDRLEVSLNCLTTALDRTDADVTIYCLDKKWKERIDAALQLKESVTELKDEDMEIDDELVWIHPDSCLKGRKGFSTTKGKLYSYFEGTKFHQAAKDMAEIKVLFPNDQESNEQLCAYILGETMEAIREKCPVDHNPSSSPPKTLPCLCMYAMTPERVHRLRSNNVKEVTVCSSTPLPKHKIKNVQKVQCTKVVLFNPHTPAFVPARKYIEVPEQPTAPPAQAEEAPEVVATPSPSTADNTSLDVTDISLDMDDSSEGSLFSSFSGSDNSITSMDSWSSGPSSLEIVDRRQVVVADVHAVQEPAPIPPPRLKKMARLAAARKEPTPPASNSSESLHLSFGGVSMSLGSIFDGETARQAAVQPLATGPTDVPMSFGSFSDGEIDELSRRVTESEPVLFGSFEPGEVNSIISSRSAVSFPLRKQRRRRRSRRTEYZLTGVGG

SEQ ID NO: 7 - DNA, 1830 bp, Sindbis virus
tacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaa

SEQ ID NO: 8 - protein, 610 aa, Sindbis virus
YIFSTDTGPGHLQKKSVLQNQLTEPTLERNVLERIHAPVLDTSKEEQLKLRYQMMPTEANKSRYQSRKVENQKAITTERLLSGLRLYNSATDQPECYKITYPKPLYSSSVPANYSDPQFAVAVCNNYLHENYPTVASYQITDEYDAYLDMVDGTVACLDTATFCPAKLRSYPKKHEYRAPNIRSAVPSAMQNTLQNVLIAATKRNCNVTQMRELPTLDSATFNVECFRKYACNDEYWEEFARKPIRITTEFVTAYVARLKGPKAAALFAKTYNLVPLQEVPMDRFVMDMKRDVKVTPGTKHTEERPKVQVIQAAEPLATAYLCGIHRELVRRLTAVLLPNIHTLFDMSAEDFDAIIAEHFKQGDPVLETDIASFDKSQDDAMALTGLMILEDLGVDQPLLDLIECAFGEISSTHLPTGTRFKFGAMMKSGMFLTLFVNTVLNVVIASRVLEERLKTSRCAAFIGDDNIIHGVVSDKEMAERCATWLNMEVKIIDAVIGERPPYFCGGFILQDSVTSTACRVADPLKRLFKLGKPLPADDEQDEDRRRALLDETKAWFRVGITGTLAVAVTTRYEVDNITPVLLALRTFAQSKRAFQAIRGEIKHLYGGPK

SEQ ID NO: 9 - DNA, 2391 bp, Artificial Sequence
  fusion protein of Sindbis virus nsp3 with GFP
gcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtggg

SEQ ID NO: 10 - protein, 797 aa, Artificial sequence
  fusion protein of Sindbis virus nsp3 with GFP
APSYRTKRENIADCQEEAVVNAANPLGRPGEGVCRAIYKRWPTSFTDSATETGTARMTVCLGKKVIHAVGPDFRKHPEAEALKLLQNAYHAVADLVNEHNIKSVAIPLLSTGIYAAGKDRLEVSLNCLTTALDRTDADVTIYCLDKKWKERIDAALQLKESVTELKDEDMEIDDELVWIHPDSCLKGRKGFSTTKGKLYSYFEGTKFHQAAKDMAEIKVLFPNDQESNEQLCAYILGETMEAIREKCPVDHNPSSSPPKTLPCLCMYAMTPERVHRLRSNNVKEVTVCSSTPLPKHKIKNVQKVQCTKVVLFNPHTPAFVPARKYIEVPEQPTAPPAQAEEAPEVVATPSPSTADNTSLDVTDISLDMDDSSEGSLFSSFSGSDNSITSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKTSMDSWSSGPSSLEIVDRRQVVVADVHAVQEPAPIPPPRLKKMARLAAARKEPTPPASNSSESLHLSFGGVSMSLGSIFDGETARQAAVQPLATGPTDVPMSFGSFSDGEIDELSRRVTESEPVLFGSFEPGEVNSIISSRSAVSFPLRKQRRRRRSRRTEYZLTGVGG

SEQ ID NO: 11 - DNA, 12426 bp, Artificial sequence
  Sindbis virus Toto1101 plasmid with nsp3/GFP fusion
attgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccatgaatagaggattctttaacatgctcggccgccgccccttcccggcccccactgccatgtggaggccgcggagaaggaggcaggcggccccgatgcctgcccgcaacgggctggcttctcaaatccagcaactgaccacagccgtcagtgccctagtcattggacaggcaactagacctcaacccccacgtccacgcccgccaccgcgccagaagaagcaggcgcccaagcaaccaccgaagccgaagaaaccaaaaacgcaggagaagaagaagaagcaacctgcaaaacccaaacccggaaagagacagcgcatggcacttaagttggaggccgacagattgttcgacgtcaagaacgaggacggagatgtcatcgggcacgcactggccatggaaggaaaggtaatgaaacctctgcacgtgaaaggaaccatcgaccaccctgtgctatcaaagctcaaatttaccaagtcgtcagcatacgacatggagttcgcacagttgccagtcaacatgagaagtgaggcattcacctacaccagtgaacaccccgaaggattctataactggcaccacggagcggtgcagtatagtggaggtagatttaccatccctcgcggagtaggaggcagaggagacagcggtcgtccgatcatggataactccggtcgggttgtcgcgatagtcctcggtggcgctgatgaaggaacacgaactgccctttcggtcgtcacctggaatagtaaagggaagacaattaagacgaccccggaagggacagaagagtggtccgcagcaccactggtcacggcaatgtgtttgctcggaaatgtgagcttcccatgcgaccgcccgcccacatgctatacccgcgaaccttccagagccctcgacatccttgaagagaacgtgaaccatgaggcctacgataccctgctcaatgccatattgcggtgcggatcgtctggcagaagcaaaagaagcgtcattgacgactttaccctgaccagcccctacttgggcacatgctcgtactgccaccatactgtaccgtgcttcagccctgttaagatcgagcaggtctgggacgaagcggacgataacaccatacgcatacagacttccgcccagtttggatacgaccaaagcggagcagcaagcgcaaacaagtaccgctacatgtcgcttaagcaggatcacaccgttaaagaaggcaccatggatgacatcaagattagcacctcaggaccgtgtagaaggcttagctacaaaggatactttctcctcgcaaaatgccctccaggggacagcgtaacggttagcatagtgagtagcaactcagcaacgtcatgtacactggcccgcaagataaaaccaaaattcgtgggacgggaaaaatatgatctacctcccgttcacggtaaaaaaattccttgcacagtgtacgaccgtctgaaagaaacaactgcaggctacatcactatgcacaggccgagaccgcacgcttatacatcctacctggaagaatcatcagggaaagtttacgcaaagccgccatctgggaagaacattacgtatgagtgcaagtgcggcgactacaagaccggaaccgtttcgacccgcaccgaaatcactggttgcaccgccatcaagcagtgcgtcgcctataagagcgaccaaacgaagtgggtcttcaactcaccggacttgatcagacatgacgaccacacggcccaagggaaattgcatttgcctttcaagttgatcccgagtacctgcatggtccctgttgcccacgcgccgaatgtaatacatggctttaaacacatcagcctccaattagatacagaccacttgacattgctcaccaccaggagactaggggcaaacccggaaccaaccactgaatggatcgtcggaaagacggtcagaaacttcaccgtcgaccgagatggcctggaatacatatggggaaatcatgagccagtgagggtctatgcccaagagtcagcaccaggagaccctcacggatggccacacgaaatagtacagcattactaccatcgccatcctgtgtacaccatcttagccgtcgcatcagctaccgtggcgatgatgattggcgtaactgttgcagtgttatgtgcctgtaaagcgcgccgtgagtgcctgacgccatacgccctggccccaaacgccgtaatcccaacttcgctggcactcttgtgctgcgttaggtcggccaatgctgaaacgttcaccgagaccatgagttacttgtggtcgaacagtcagccgttcttctgggtccagttgtgcatacctttggccgctttcatcgttctaatgcgctgctgctcctgctgcctgccttttttagtggttgccggcgcctacctggcgaaggtagacgcctacgaacatgcgaccactgttccaaatgtgccacagataccgtataaggcacttgttgaaagggcagggtatgccccgctcaatttggagatcactgtcatgtcctcggaggttttgccttccaccaaccaagagtacattacctgcaaattcaccactgtggtcccctccccaaaaatcaaatgctgcggctccttggaatgtcagccggccgctcatgcagactatacctgcaaggtcttcggaggggtctacccctttatgtggggaggagcgcaatgtttttgcgacagtgagaacagccagatgagtgaggcgtacgtcgaattgtcagcagattgcgcgtctgaccacgcgcaggcgattaaggtgcacactgccgcgatgaaagtaggactgcgtattgtgtacgggaacactaccagtttcctagatgtgtacgtgaacggagtcacaccaggaacgtctaaagacttgaaagtcatagctggaccaatttcagcatcgtttacgccattcgatcataaggtcgttatccatcgcggcctggtgtacaactatgacttcccggaatatggagcgatgaaaccaggagcgtttggagacattcaagctacctccttgactagcaaggatctcatcgccagcacagacattaggctactcaagccttccgccaagaacgtgcatgtcccgtacacgcaggcctcatcaggatttgagatgtggaaaaacaactcaggccgcccactgcaggaaaccgcacctttcgggtgtaagattgcagtaaatccgctccgagcggtggactgttcatacgggaacattcccatttctattgacatcccgaacgctgcctttatcaggacatcagatgcaccactggtctcaacagtcaaatgtgaagtcagtgagtgcacttattcagcagacttcggcgggatggccaccctgcagtatgtatccgaccgcgaaggtcaatgccccgtacattcgcattcgagcacagcaactctccaagagtcgacagtacatgtcctggagaaaggagcggtgacagtacactttagcaccgcgagtccacaggcgaactttatcgtatcgctgtgtgggaagaagacaacatgcaatgcagaatgtaaaccaccagctgaccatatcgtgagcaccccgcacaaaaatgaccaagaatttcaagccgccatctcaaaaacatcatggagttggctgtttgcccttttcggcggcgcctcgtcgctattaattataggacttatgatttttgcttgcagcatgatgctgactagcacacgaagatgaccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttc